(19) United States
(12) Patent Application Publication
(10) Pub. No.: US 2020/0020434 A1
(43) Pub. Date: Jan. 16, 2020

(54) INFORMATION COLLECTION PROCESSING APPARATUS, INFORMATION COLLECTION PROCESSING METHOD, AND RECORDING MEDIUM

(71) Applicant: KONICA MINOLTA, INC., Tokyo (JP)

(72) Inventors: Hirotake MINAMI, Fuchu-shi (JP); Shinsuke KATSUHARA, Kodaira-shi (JP); Kenji OHNUMA, Hino-shi (JP); Hidetake TEZUKA, Tachikawa-shi, Tokyo (JP)

(21) Appl. No.: 16/445,705

(22) Filed: Jun. 19, 2019

(30) Foreign Application Priority Data
Jul. 12, 2018 (JP) 2018-131951

(51) Int. Cl. G16H 30/20

(52) U.S. Cl. CPC G16H 30/20

(57) ABSTRACT

An information collection processing apparatus which can efficiently collect desired medical information without excess or deficiency, an information collection processing method and a program are provided. The information collection processing apparatus includes an information extractor which extracts medical information from a terminal apparatus which possesses the medical information including a plurality of pieces of supplementary information on the basis of collection side conditions in which medical information to be collected is set and provision side conditions in which medical information which can be provided is set, an information processor which performs treatment processing including processing of deleting at least one piece of the supplementary information among a plurality of pieces of the supplementary information included in the medical information extracted by the information extractor, and an information storage 6 which stores the medical information subjected to the treatment processing.

### CROSS REFERENCE TO RELATED APPLICATION

The present application claims priority under 35 U.S.C. § 119 to Japanese Application No. 2018-131951 filed Jul. 12, 2018, the entire content of which is incorporated herein by reference.

### BACKGROUND

### Technological Field

The present invention relates to an information collection processing apparatus, an information collection processing method, and a recording medium.

### Description of the Related Art

Recently, in the field of medicine, concerning diagnostic imaging, or the like, which is performed by a person, there is trend of supporting diagnosis using machine learning such as AI or causing technology using machine learning to perform a diagnostic action itself.

The machine learning is a technique which causes machine to learn patterns and correlation of data using a large amount of data so as to enable the machine to perform various kinds of detection, or the like.

To perform detection with high accuracy using machine learning in the field of medicine, it is necessary to collect a number of pieces of medical image data as data to be used for learning, information regarding diagnosis, or the like, and information regarding patients who visit medical institutions (that is, facilities such as hospitals) (these will be also collectively referred to as “medical information”), or the like.

Concerning this point, Japanese Patent Laid-Open No. 2000-157495 proposes an image collection and display apparatus which is configured with an image collector, an image storage and an image display, and which can associate collected images with image numbers in accordance with a specific rule and can display images in accordance with a predetermined display plan, as an apparatus which collects and displays image data.

To collect data to be used for learning, it is required to collect data widely from a plurality of medical institutions such as hospitals and clinics for a plurality of patients.

However, the apparatus disclosed in Japanese Patent Laid-Open No. 2000-157495 is an apparatus which enables images, or the like, to be displayed at appropriate display positions in appropriate display order when the images, or the like, photographed for examinations are collected and displayed, and does not assume collection of a large amount of data regarding a plurality of patients.

Further, the collected data is assumed to be used for diagnosis, or the like, of examination results, and the data is not assumed to be utilized outside of medical institutions.

Information (medical information) regarding patients acquired at medical institutions include a number of pieces of highly personal information. Therefore, in terms of personal information protection, even in the case where data is digital data via a network, to take the medical information outside of the medical institutions, it is necessary to anonymize the data or perform anonymization processing on the data. However, there is a problem that it is extremely troublesome to perform such treatment and processing on a large amount of data.

Further, because types, characteristics, handling of personal information, or the like, of data possessed by medical institutions are different for each facility, in the case where data is collected over a plurality of facilities, there is also a problem that it is troublesome to efficiently collect only necessary information.

### SUMMARY

The present invention has been made in view of the above-described circumstances, and is directed to providing an information collection processing apparatus which can efficiently collect desired medical information without excess or deficiency, an information collection processing method and a recording medium.

To achieve at least one of the abovementioned objects, according to a first aspect of the present invention, an information collection processing apparatus reflecting one aspect of the present invention comprises

a hardware processor which extracts medical information from a terminal apparatus which possesses the medical information including a plurality of pieces of supplementary information on the basis of a collection side condition in which medical information to be collected is set and a provision side condition in which medical information which can be provided is set, and performs treatment processing of deleting at least one piece of the supplementary information among a plurality of pieces of the supplementary information included in the extracted medical information, and

an information storage which stores the medical information subjected to the treatment processing.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a schematic entire configuration diagram illustrating relationship between an information collection processing apparatus and a terminal apparatus in the present embodiment;

FIG. 2 is a diagram illustrating an example of a provision side condition setting screen displayed at a display of the terminal apparatus;

FIG. 3 is a schematic block diagram illustrating a main part of a control configuration of the information collection processing apparatus in the present embodiment;

FIG. 4 is a diagram illustrating an example of a collection side condition setting screen displayed at the display of the information collection processing apparatus; and

FIG. 5 is a flowchart illustrating an information collection processing method in the present embodiment.

### DETAILED DESCRIPTION OF EMBODIMENTS

Hereinafter, one or more embodiment of the present invention will be described with reference to the drawings. However, the scope of the invention is not limited to the disclosed embodiments.

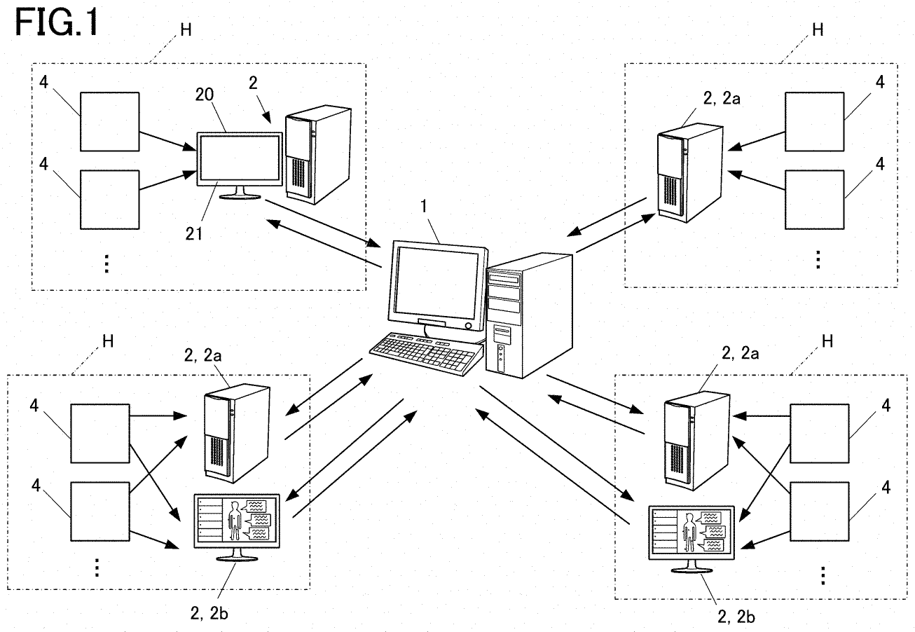

An embodiment of an information collection processing apparatus, an information collection processing method and a recording medium according to the present invention will be described below with reference to FIG. 1 to FIG. 5.

### [Entire Configuration Including Information Collection Processing Apparatus]

FIG. 1 is a schematic entire configuration diagram illustrating aspect where an information collection processing apparatus is connected to a terminal apparatus within each facility and exchanges information.

An information collection processing apparatus 1 in the present embodiment is an apparatus which collects necessary medical information among medical information accumulated in a terminal apparatus 2 within a facility H which is a medical institution. The collected medical information is stored in an information storage 6 (a medical information storage region 62 within the information storage 6) which will be described later.

In the present embodiment, the medical information to be stored in the information storage 6 is image data which can be acquired at the facility H which is a medical institution, and information regarding patients including diagnosis information.

More specifically, the medical information broadly includes various kinds of medical related information accumulated in medical institutions, such as image data of various kinds of medical images such as X-ray images, ultrasonic images, X-ray computer tomography images, magnetic resonance images and nuclear medicine images (tomographic images such as SPECT (Single Photon Emission Tomography) and PET (Positoron Emission Tomography)), electronic health record information, computer-aided diagnosis system (CAD) information, patient information included in receipt information, or the like, and information of diagnosis results by doctors.

The terminal apparatus 2 is an apparatus which possesses medical information and becomes a provider of the medical information.

Specifically, for example, as illustrated in FIG. 1, the terminal apparatus 2 is a PACS terminal 2a which is connected to various kinds of medical systems 4 provided within the facility H and to which the medical information acquired by the various kinds of medical systems 4 is transmitted and accumulated, an electronic health record terminal 2b in which each item is automatically or manually input by reception, or the like, of patients, or the like.

The medical system 4 is, for example, a medical image system configured with various kinds of modalities (not illustrated) and a console for photographing which controls photographing operation by the modalities (not illustrated).

The modality photographs a portion to be photographed, or the like, of a patient, and converts the photographed image into digital data to generate image data (medical image data), or the like, as medical information for medical use.

While, as the modalities, for example, a CR (Computed Radiography, X-ray plain radiography) device, a mammography device, a CT (Computed Tomography, computer tomography) device, an ultrasonic diagnosis device, an endoscopic device, or the like, (none of them is illustrated) are assumed, the modalities are not limited to these.

Note that, while, in FIG. 1, two pairs of medical systems 4 each configured with a modality and a console for photographing are illustrated for each facility H, the number of the medical systems 4 provided within the facility H is not limited to this, and one or three or more medical systems 4 may be provided.

The medical system 4 (that is, a modality and a console for photographing connected to the modality), the PACS terminal 2a and the electronic health record terminal 2b, or the like, which are terminal apparatuses 2 are connected to a network inside a hospital, which is not illustrated, via a switching hub, or the like, which is not illustrated, so as to be able to transmit and receive data via the network.

The respective apparatuses connected via the network typically conform to, for example, DICOM (Digital Image and Communications in Medicine) standards which are communication standards handling medical-related data, and communication between the respective apparatuses is performed in accordance with the DICOM.

Further, various kinds of supplementary information are accompanied by medical information generated in accordance with the DICOM standards.

For example, in the case where the medical system 4 which generates image data (medical image data) of a medical image as the medical information complies with the DICOM standards, the image data is stored in a DICOM file format in accordance with the DICOM standards. The DICOM file includes an image part and a supplementary information part, and actual data of the medical image is written in the image part, and various kinds of supplementary information regarding the medical image are written in the supplementary information part.

The supplementary information includes, for example, patient information, examination information and image detailed information.

Here, the patient information includes various kinds of information regarding a patient of a medical image such as patient identification information (for example, a patient ID) for identifying the patient, patient name, sex and birth date.

Further, the examination information includes various kinds of information regarding examination such as examination identification information (for example, an examination ID) for identifying examination, examination date, a type of the modality, an examined portion and a doctor-in-charge. Note that the examination information may also include information regarding photographing conditions, positioning, or the like, upon acquisition of the image data.

The image detailed information includes various kinds of information regarding a medical image such as an image ID, image generation time, file path name indicating a location where the medical image is stored.

The respective pieces of supplementary information are managed with tags in accordance with the DICOM standards, and it is possible to determine specific content of the respective pieces of supplementary information (that is, whether the supplementary information is a patient ID, patient name or a type of the modality) by referring to the tags.

These kinds of medical information (that is, the image data, the supplementary information, or the like) are transmitted to the terminal apparatus 2 such as the PACS terminal 2a and stored.

The electronic health record terminal 2b is the terminal apparatus 2 which receives input of and possesses electronic health record information such as personal information and a medical interview result of a patient, which are acquired upon reception of the patient, and a diagnosis result by a doctor.

The electronic health record information is information regarding a patient, and is medical information, and, specifically, includes, for example, respective items of a patient ID, patient name, sex, age, body weight, body height, a body temperature, a medical interview result, a diagnosis result by a doctor, fill-in date, or the like. Note that the electronic health record information is not limited to these.

In the present embodiment, at the terminal apparatus 2 on an information provision side (that is, the terminal apparatus 2 which becomes a provider of information and which possesses medical information), provision side conditions in which medical information which can be provided is set in advance are determined.

The provision side conditions are conditions which specify medical information which can be provided to the information collection processing apparatus 1 at each facility H.

For example, the terminal apparatus 2 includes a monitor device 20 which includes a display 21 configured with an LCD (Liquid Crystal Display), a CRT (Cathode Ray Tube), and the like, an operator which is not illustrated, or the like, and the provision side conditions are set from the monitor device 20 of the terminal apparatus 2. Note that, while, in FIG. 1, the monitor device 20 is illustrated only at the terminal apparatus 2 provided at the facility H in an upper left part, the monitor device 20 may be provided at all the terminal apparatuses 2.

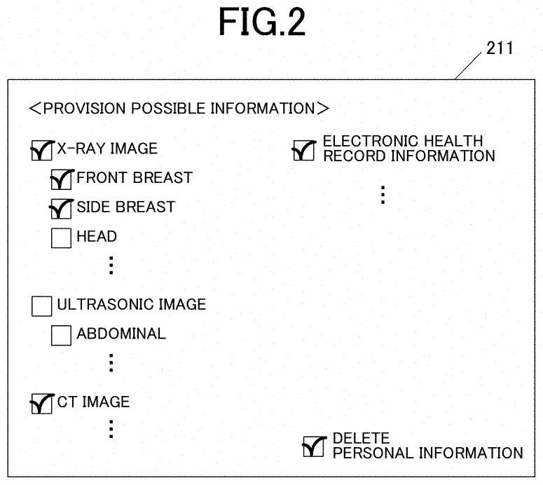

FIG. 2 is a display screen example illustrating an example of a provision possible information setting screen to be displayed at the monitor device 20 of the terminal apparatus 2.

As illustrated in FIG. 2, on the provision possible information setting screen 211, a list of medical information (such as, for example, an X-ray image of the front breast, an X-ray image of the head, an ultrasonic image and electronic health record information) acquired at the medical system 4 connected to the terminal apparatus 2 and accumulated in the terminal apparatus 2 is displayed, and, on the facility H side, medical information which may be provided outside can be selected and set at the facility among the displayed list.

In the example illustrated in FIG. 2, a list of the medical information is displayed in a state where check boxes are provided to the respective pieces of medical information, so as to allow only check boxes of the medical information which can be provided to be checked.

Further, on the provision possible information setting screen 211, information as to whether or not it is necessary to delete personal information is displayed with a check box, so that whether or not it is necessary to delete personal information can be selected and set.

In the present embodiment, the personal information is highly personal information from which it is possible to specify an individual patient, among patient information relating to the patient, and is, for example, a patient ID which is identification information (identifying indicator) of the individual patient, patient name, or the like.

Note that which information among the patient information is to be deleted as the personal information may be designated as the provision side conditions.

By a check box being checked on the provision possible information setting screen 211, the provision side conditions are set.

The set provision side conditions are read to the information collection processing apparatus 1 side when the information collection processing apparatus 1 is connected to the terminal apparatus 2, and are stored in a condition information storage region 61 of the information storage 6 which will be described later.

Note that a way of setting the provision side conditions is not limited to the way described here. Further, layout, or the like, of the provision possible information setting screen 211 is not limited to that illustrated in FIG. 2.

### [Configuration of Information Collection Processing Apparatus]

The information collection processing apparatus 1 receives medical information from the terminal apparatus 2 such as the PACS terminal 2a and the electronic health record terminal 2b within the facility H.

Note that the information collection processing apparatus 1 may be actually provided within each facility H and may be connected to the terminal apparatus 2 such as the PACS terminal 2a and the electronic health record terminal 2b within the facility H.

In the case where on-premise operation is performed within the facility H in which the terminal apparatus 2 is provided and information accumulated in the terminal apparatus 2 is aggregated in a server, or the like, provided within the facility H, it is also possible to employ a configuration where the information collection processing apparatus 1 can take the medical information by accessing the server, or the like.

Further, in the case where medical information is uploaded to cloud, or the like, from the terminal apparatus 2 via a computer network such as the Internet, it is also possible to employ a configuration where the information collection processing apparatus 1 can take the medical information by accessing this cloud, or the like.

In this case, it is also possible to employ a configuration where information of medical institutions of the same affiliation, and medical institutions which are affiliated with each other, or the like, as well as the terminal apparatuses 2 within one facility H is uploaded to cloud, or the like, and the information collection processing apparatus 1 can collectively collect information accumulated in the terminal apparatuses 2 of a plurality of facilities H by accessing the cloud. With such a configuration, the information collection processing apparatus 1 can broadly collect a larger amount of medical information

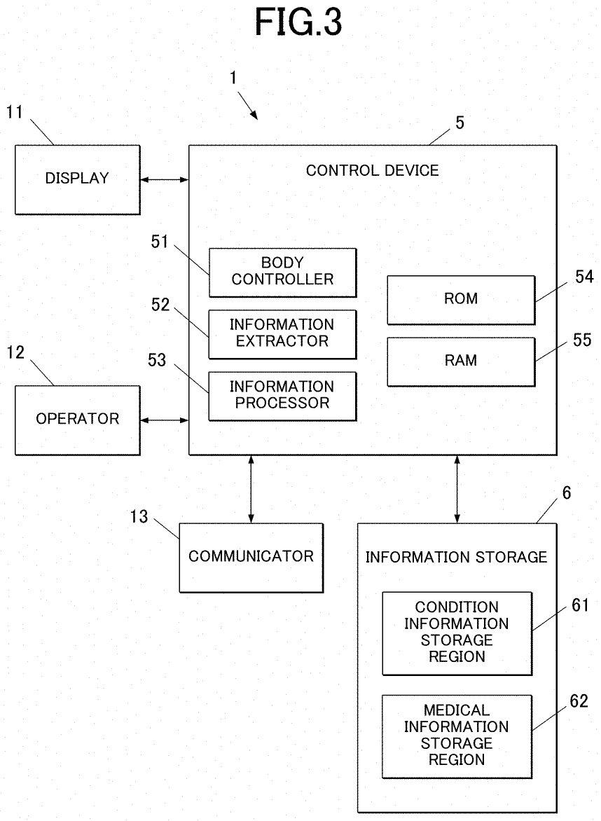

FIG. 3 is a block diagram of a main part of a control configuration of the information collection processing apparatus.

As illustrated in FIG. 3, the information collection processing apparatus 1 in the present embodiment includes a display 11, an operator 12, a communicator 13, a control device 5, an information storage 6, or the like.

The display 11 is configured with a monitor such as, for example, an LCD (Liquid Crystal Display) and a CRT (Cathode Ray Tube), and displays various kinds of screens, medical images, or the like, in accordance with an instruction of a display signal input from the control device 5.

In the present embodiment, at the information collection processing apparatus 1 which becomes an information requesting side, collection side conditions in which medical information to be collected is set in advance are determined.

The collection side conditions are conditions which specify medical information which is desired to be collected at the information collection processing apparatus 1.

The display 11 displays a collection target information setting screen 111 for setting collection target information.

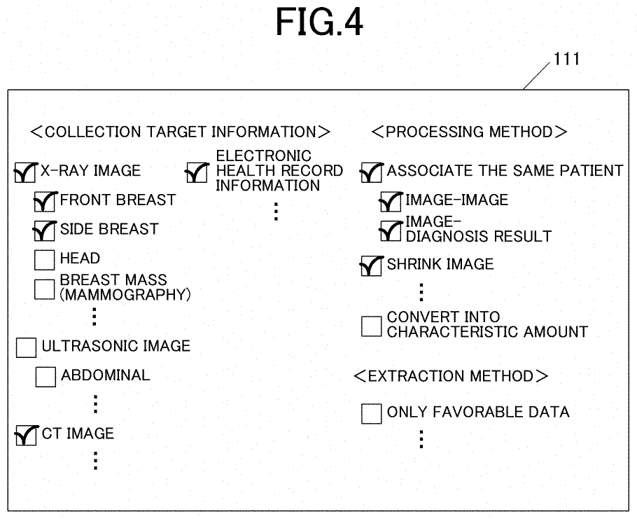

FIG. 4 is a display screen example illustrating an example of the collection target information setting screen to be displayed at the display 11 of the information collection processing apparatus 1.

As illustrated in FIG. 4, on the collection target information setting screen 111, a list of medical information (such as, for example, an X-ray image of the front breast, an X-ray image of the head, an ultrasonic image and electronic health record information) which is likely to be acquired from the terminal apparatus 2 is displayed, and an operator of the information collection processing apparatus 1 can select and set medical information which is desired to be collected with the information collection processing apparatus 1 among the displayed list.

In the example illustrated in FIG. 4, a list of the medical information is displayed in a state where check boxes are provided to the respective pieces of medical information, so as to allow only check boxes of medical information which can be provided to be checked.

Further, the information collection processing apparatus 1 of the present embodiment can perform treatment processing on medical information using a plurality of processing methods, and a list of processing methods which can be performed at an information processor 53 which will be described later is displayed on the collection target information setting screen 111 in addition to the list of medical information.

Further, in the case where there are a plurality of extraction methods of medical information to be performed by an information extractor 52 which will be described later, a list of the extraction methods is also displayed on the collection target information setting screen 111.

In the example illustrated in FIG. 4, a list of the processing methods and the extraction methods is displayed in a state where check boxes are provided to the respective processing methods and the respective extraction methods, so as to allow only check boxes of the processing method and the extraction method to be selected and set, to be checked.

By check boxes being checked on the collection target information setting screen 111, the collection side conditions are set.

The set collection side conditions are stored in the condition information storage region 61 of the information storage 6 (which will be described later) of the information collection processing apparatus 1.

Note that a way of setting the collection side conditions is not limited to the way described here. Further, layout, or the like, of the collection target information setting screen 111 is not limited to that illustrated in FIG. 4.

In the case where none of the check boxes of the processing methods and the check boxes of the extraction methods is checked, it is also possible to encourage the operator of the information collection processing apparatus 1 to select and set one processing method and one extraction method by displaying a message such as “please select an extraction method” and “please select a processing method” on the display screen, or giving a warning using sound, or the like.

Note that, concerning medical information to be collected, the collection side conditions set in advance at the information collection processing apparatus 1 which is an information requesting side are not limited to the collection side conditions set by an on-site operator at the information collection processing apparatus 1.

For example, in a factory shipment stage, or the like, the collection side conditions may be set by default by a requester who requests collection of information, and may be stored in advance in the condition information storage region 61 of the information storage 6. In this case, it is not necessary to particularly display a display screen like the collection target information setting screen 111 at the display 11.

Further, also in the case where the collection side conditions are set by default, a display screen like the collection target information setting screen 111 may be displayed to set addition, correction, or the like, of the collection side conditions afterward, and information may be collected on the basis of the added and corrected collection side conditions.

The operator 12 includes, for example, a keyboard including a cursor key, numeric keys, various kinds of function keys, or the like, a pointing device such as a mouse, or the like.

An input signal (instruction signal) input through key operation, mouse operation, or the like, with respect to the key board is output to the control device 5.

The communicator 13 includes, for example, a LAN adapter, a router, a TA (Terminal Adapter), or the like, and transmits/receives data with various kinds of devices which are not illustrated and which are connected to a network.

The control device 5 is configured with a function part configured with a CPU, or the like, which is not illustrated, and a ROM 54, a RAM 55, or the like.

In the present embodiment, the control device 5 includes function parts such as a body controller 51, an information extractor 52, an information processor 53, or the like, as illustrated in FIG. 3 from a functional viewpoint.

The respective function parts at the control device 5 are computers which realize various kinds of functions by the CPU reading various kinds of programs such as a system program and a processing program (for example, an information collection processing program) stored in the ROM 54, expanding the programs in a work area of the RAM 55 and cooperating with the expanded programs.

The body controller 51 comprehensively controls respective components of the information collection processing apparatus 1 by outputting a display signal to the display 11 to cause various kinds of display screens to be displayed, accepting an input signal input from the operator 12 to cause each component to operate so as to perform processing in accordance with the input signal, or the like.

The information extractor 52 extracts medical information from the terminal apparatus 2 such as the PACS terminal 2a and the electronic health record terminal 2b in accordance with the processing programs stored in the ROM 54.

The information extractor 52 causes the extracted medical information to be stored in a medical information storage region 62 of the information storage 6.

In the present embodiment, the information extractor 52 extracts medical information from the terminal apparatus 2 which possesses medical information including a plurality of pieces of supplementary information on the basis of the collection side conditions in which the medical information to be collected at the information collection processing apparatus 1 is set and the provision side conditions in which the medical information which can be provided at the terminal apparatus 2 which possesses the information is set.

In the present embodiment, extracting medical information on the basis of the collection side conditions and the provision side conditions means collating the both conditions to extract medical information which satisfy the both conditions.

As a specific extraction method by the information extractor 52, only in the case where the medical information to be collected matches the medical information which can be provided (in a case of exact match), the medical information which matches each other may be collected, or if the medical information at least partly matches each other, the medical information in a range where the medical information matches each other may be collected.

Further, in the case where the medical information to be collected matches the medical information which can be provided in at least one or more pieces, all the medical information which can be provided may be acquired, or only part of the medical information may be acquired.

Still further, for example, an essential acquisition information check button for designating medical information which should be necessarily collected (essential acquisition information) may be provided on the collection target information setting screen 111 for setting the collection side conditions, and, in the case where the medical information which is made the essential acquisition information matches the medical information which can be provided at the provision side, all of the medical information which can be provided or only the medical information which is made the essential acquisition information may be acquired.

Note that the extraction method by the information extractor 52 is not limited to the example described here, and various kinds of methods of conditioning, or the like, may be used as appropriate to select and extract only desired medical information.

The medical information collected by the information collection processing apparatus 1 of the present embodiment is used in predetermined application and purpose such as being used as various kinds of learning data, and, even if data which is not appropriate in light of such application and purpose, the data becomes useless.

Concerning this point, in the case where various kinds of conditioning are performed in a stage where information is extracted by the information extractor 52, it is possible to efficiently collect only useful data in light of application and purpose. By this means, it is possible to realize reduction in a processing period, suppress an amount of data to be collected and reduce load on the medical information storage region 62.

Specifically, for example, as illustrated in FIG. 4, as the extraction method, conditioning may be performed so that only favorable data is extracted as desired medical information.

Here, in the case where the medical information is image data, for example, the favorable data is data in which there is no defect in photographing conditions and positioning upon acquisition of the image data.

Further, it is also possible to learn a classifier for sorting whether or not the data is desired medical information in advance, and sort and extract only desired medical information using this classifier. In this case, for example, after the medical information is extracted in a range where the medical information to be collected matches the medical information which can be provided, the medical information is sorted using the classifier, and only the medical information sorted as favorable data is acquired (that is, caused to be stored in the medical information storage region 62).

For example, in the case where the medical information is image data, a classifier which classifies whether or not the data is favorable data as an image in terms of photographing conditions, positioning, or the like, upon acquisition of image data is learned in advance, and cleansing which selects favorable data is performed using this classifier (classifier for classifying whether data is favorable data or poor data).

The classifier for classifying whether data is favorable data or poor data can select favorable data by utilizing a transfer learning model such as AlexNet and GoogleNet which are typically used in a classification problem in deep learning (please see, for example, https://www.cs.toronto.edu/˜kriz/imagenet_classification_with_deep_convolutional.pdf for a specific selection method, or the like).

By using such machine learning (deep learning), it is possible to exclude poor data in which there is an abnormality in photographing conditions, a defect in positioning, or the like, from medical information to be acquired by the information collection processing apparatus 1. Therefore, it is possible to efficiently collect only favorable medical information and suppress an amount of data to be stored in the medical information storage region 62.

Note that the extraction method by the information extractor 52 is not limited to the method using machine learning (deep learning). For example, information regarding the photographing conditions (such as, for example, an amount of X-ray in volts used to perform exposure) and information regarding positioning (such as an angle at which photographing is performed and whether or not displacement of a position, or the like, occurs upon photographing) may be included in examination information as supplementary information of the image data. In the case where these kinds of information accompany the image information as the examination information, the information extractor 52 may judge whether or not the data is favorable data acquired under appropriate conditions by also taking into account information such as, for example, a photographing portion and information regarding the photographing conditions and positioning.

The information processor 53 performs treatment processing including processing of deleting at least one piece of supplementary information among a plurality of pieces of the supplementary information included in the medical information extracted by the information extractor 52 in accordance with the processing programs stored in the ROM 54.

The information processor 53 causes the medical information subjected to the treatment processing in the medical information storage region 62 of the information storage 6.

In the present embodiment, the information processor 53 has a plurality of processing methods. For example, in the case where one or a plurality of processing methods are selected and set on the collection target information setting screen 111, the information processor 53 performs treatment processing on the medical information using the selected and set processing methods.

Note that the information processor 53 does not have to have a plurality of processing methods, and a case where the information processor 53 has one processing method is not excluded from the scope of the present invention.

First, the treatment processing to be performed by the information processor 53 includes processing of deleting at least one piece of supplementary information among a plurality of pieces of supplementary information included in the medical information as described above. In the present embodiment, “at least one piece of supplementary information” is at least personal information of a patient.

In the case where the medical information is image data in accordance with the DICOM standards, personal information, or the like, accompanies the data as the supplementary information as described above, and the respective pieces of supplementary information are managed with predetermined tags.

The information processor 53 selects information from which it is possible to specify an individual patient (personal information such as, for example, patient name and a patient ID), included in the medical information by referring to a tag, and deletes these kinds of information to process the data to generate data which does not include the personal information.

Note that, because the patient name and the patient ID are information from which it is possible to specify an individual patient, while the patient name and the patient ID have to be necessarily deleted when the medical information is taken outside of the facility H, concerning the other information, a range of information to be deleted may be selected and set as appropriate. For example, while age and sex are also personal information of a patient, because these kinds of information are not information from which it is possible to specify an individual patient if the information is separated from the patient name and the patient ID, and there are diseases, or the like, which characteristically appear depending on age and sex, there is also a case where it is meaningful to leave age and sex while age and sex are associated with image data, or the like. Therefore, which information is to be deleted (removed) as personal information may be determined in accordance with purpose, or the like, for collecting medical information.

Further, the information processor 53 may be able to cut out only data regarding a lesion position specified in accordance with lesion information.

The lesion information is information of a portion, or the like, where there is a lesion portion among the body of the patient.

In the case where the medical information is image data, a lesion portion (which will be also referred to as a region of interest, a region to be examined or ROI (Region of Interest)) can be often specified by referring to supplementary information accompanied by the image data, the electronic health record information, computer-aided diagnosis system (CAD) information, reception information, or the like.

Therefore, in the case where it is possible to specify a lesion portion by referring to these kinds of information, and the lesion portion is limited to part of the image data, the information processor 53 may perform processing of cutting out only part of the image data such as a portion where there is the lesion portion, on the medical information and causes only the cut-out information (data regarding the lesion position) to be stored in the medical information storage region 62 of the information storage 6.

Because, if the data is only part of the image data (medical information), there is also a case where the data can be used for learning to identify the lesion, it is meaningful to store only cutout information (data regarding the lesion position) as collection of the medical information.

Then, by only a necessary portion among the medical information being stored in the medical information storage region 62 of the information storage 6 in this manner, it is possible to suppress an amount of data to be stored in the information storage 6.

Further, in this case, if only a necessary portion among the medical information is stored separately from the personal information of a patient such as patient name, it is possible to collect the medical information while reliably preventing leakage of personal information.

Note that, in the case where the personal information is removed from the medical information through cutting-out processing of data regarding the lesion position, which is performed by the information processor 53 in this manner, the cutting-out processing becomes treatment processing of deleting at least one piece of supplementary information.

Further, the information processor 53 may convert medical information (for example, image data) which is extracted by the information extractor 52 among the medical information (for example, image data) possessed by the terminal apparatus 2 which is a provider, into an irreversible characteristic amount (parameter) through learning within the facility H in which the terminal apparatus 2 on the information provision side is provided. In this case, the characteristic amount (parameter) is stored in the medical information storage region 62 of the information storage 6.

To convert the medical information into the characteristic amount, the image data in the medical information and information (label) as to which portion (for example, the breast) is photographed in the image data are required.

Note that a method for converting the image data, the portion information (label), or the like, into the characteristic amount is not particularly limited. For example, learning may be performed using machine learning such as deep learning. A method of machine learning, a network to be used for deep learning, or the like, may be arbitrarily selected or may be fixed. Because a difference occurs in a way of learning and a learning result depending on what is selected as a method of machine learning, a network to be used for deep learning, or the like, it is preferable to select an appropriate method of machine learning, network, or the like, as appropriate. As a way of selection, for example, a selection screen, or the like, which is not illustrated, may be displayed at the display 11, and the operator of the information collection processing apparatus 1 may select a method, a network, or the like, in accordance with purpose, or the like, of information collection.

In this manner, by the medical information being converted into a characteristic amount (extracted from the image data, or the like, through learning) which does not include image data, or the like, within the facility H, it is possible to accumulate information which does not include personal information and which can be safely taken outside of the facility H in the medical information storage region 62, so that it is possible to efficiently collect information.

Further, it is possible to suppress an amount of data to be accumulated in the medical information storage region 62 compared to a case where the image data itself is accumulated.

In this manner, in the case where personal information is removed from the medical information through conversion processing into the characteristic amount to be performed by the information processor 53, the conversion processing becomes treatment processing of deleting part of information such as personal information of the medical information.

Note that the information processor 53 may cut out only a lesion portion (a region of interest, a region to be examined, ROI) from the image data within the facility H, may cause the lesion portion to be stored in the medical information storage region 62 after portion information (label) is provided to this lesion portion (ROI), and, then, may convert this into a characteristic amount on the basis of the lesion portion (ROI) and the portion information (label) thereof. In this case, the portion information (label) is preferably encrypted.

Further, the information processor 53 may perform processing of associating respective pieces of medical information regarding the same patient with each other.

In the present embodiment, the information processor 53 performs treatment processing of deleting personal information from which it is possible to specify a patient such as a patient ID concerning the medical information.

However, there is a case where it is meaningful to associate respective pieces of medical information such as image data for the same patient in the case where the collected information is used as learning data, or the like, of machine learning.

Therefore, the information processor 53 may provide unique identification information (identifying indicator) which is different from a patient ID, or the like, and which is not associated with an individual patient, to the respective pieces of medical information to associate the respective pieces of medical information regarding the same patient with each other. In this case, the same identification information (identifying indicator) may be provided to the medical information regarding the same patient, or association information, or the like, which indicates respective pieces of identification information provided to the medical information regarding the same patient may be stored while different types of identification information (identifying indicators) are provided to the respective pieces of medical information.

By this means, even if personal information is deleted, it is possible to distinguish medical information of the same patient, so that it is possible to efficiently collect medical information which is effective for later learning in a safe form which does not allow an individual to be specified.

Note that a timing at which the information processor 53 performs treatment processing of deleting personal information from which it is possible to specify a patient on the medical information may be before the medical information is taken outside of the facility H, and is not particularly limited.

For example, extraction processing of the medical information by the information extractor 52 is performed, and even if the extracted medical information is stored in the medical information storage region 62, the medical information may include personal information until the medical information is taken outside of the facility H.

By this means, even in the case where additional examination and photographing are performed for the same patient, and medical information is added, it is possible to associate and store the medical information regarding the same patient on the basis of personal information such as a patient ID.

For example, in the case where, after image data concerning one patient is extracted by the information extractor 52 as the medical information and stored in the medical information storage region 62, diagnosis information (diagnosis result), or the like, for the same patient is added and input to the electronic health record terminal 2b, or the like, or in the case where image data is acquired by photographing being newly performed, it is preferable that a new diagnosis result and image data, and modality information which photographs the image, management number, or the like, are associated with image data stored beforehand in accordance with the patient ID, or the like, and are stored and accumulated in the medical information storage region 62.

In this manner, in the case where new diagnosis information (diagnosis result), image data, or the like, for the same patient are added, information within the medical information storage region 62 may be able to be updated.

Then, in this case, the information processor 53 deletes personal information such as a patient ID from the medical information before the medical information is taken outside of the facility H.

By this means, it is possible to maintain association of the medical information until the medical information is taken outside of the facility H, so that it is possible to make information collection more efficient.

Further, in the case where the medical information is image data, the information processor 53 may be able to reduce image data.

Note that a method for reducing image data is not particularly limited, and various kinds of methods can be used.

By reducing image data, it is possible to reduce an amount of data to be stored in the medical information storage region 62.

Note that the information processor 53 may reduce image data for all images, or may determine whether or not to reduce image data (including whether to make image data high-resolution data or low-resolution data, or the like), a reduction rate, or the like, in the case where the image data is reduced, in accordance with image characteristics of the image data.

The image characteristics include a portion of the body, which is photographed, a lesion which is made a target of photographing, or the like.

For example, in the case where the breast mass is photographed by a mammography device, in the case where the photographing is performed for confirming a tumor mass portion, because a lesion portion of a tumor mass is large, whether or not there is a tumor mass can be confirmed even if image data is reduced (resolution is made lower). In contrast, because calcification occurring at the breast mass is a fine lesion of approximately 100μ, if an image is shrunk, it is difficult to detect the lesion, and, therefore, it is preferable to store the image data while image quality is maintained as high as possible (high resolution).

Therefore, in the case where the image characteristics are a tumor mass of the breast mass photographed by the mammography device, the image data is made low-resolution image data with a small data amount by reducing image data to ½, or the like, while, in the case where the image characteristics are calcification of the breast mass, the image data with high resolution is stored in the medical information storage region 62 without the image data being reduced.

Note that the image characteristics are determined on the basis of the lesion information.

The lesion information may be, for example, diagnosis information stored in the electronic health record terminal 2b, or lesion information acquired from a diagnosis result by a doctor, or may be lesion information acquired from a result obtained in a computer-aided diagnosis system such as a CAD, or the like.

The information processor 53 performs treatment processing on the medical information such as image data with reference to these kinds of information.

In the case where the information processor 53 reduces the image data, criteria for judgment as to whether or not to reduce the image data, or the like, are not limited to those in accordance with the image characteristics.

For example, the information processor 53 may determine whether or not to reduce the image data, a reduction rate, or the like, in the case where the image data is reduced on the basis of application and purpose of the image data, such as application of information collected by the information collection processing apparatus 1.

For example, the information processor 53 may make judgement that, in the case where the image data is used as learning data for learning to detect whether or not there is calcification of the breast mass, the original image data is stored as is as far as possible without the image data being reduced, while, in the case where the image data is used as learning data for learning to detect a position, a range, or the like, of a predetermined organ from the image, the image data is reduced, and the image data with a small data amount is stored, or the like.

### [Operation of Information Collection Processing Apparatus (Information Collection Processing Method)]

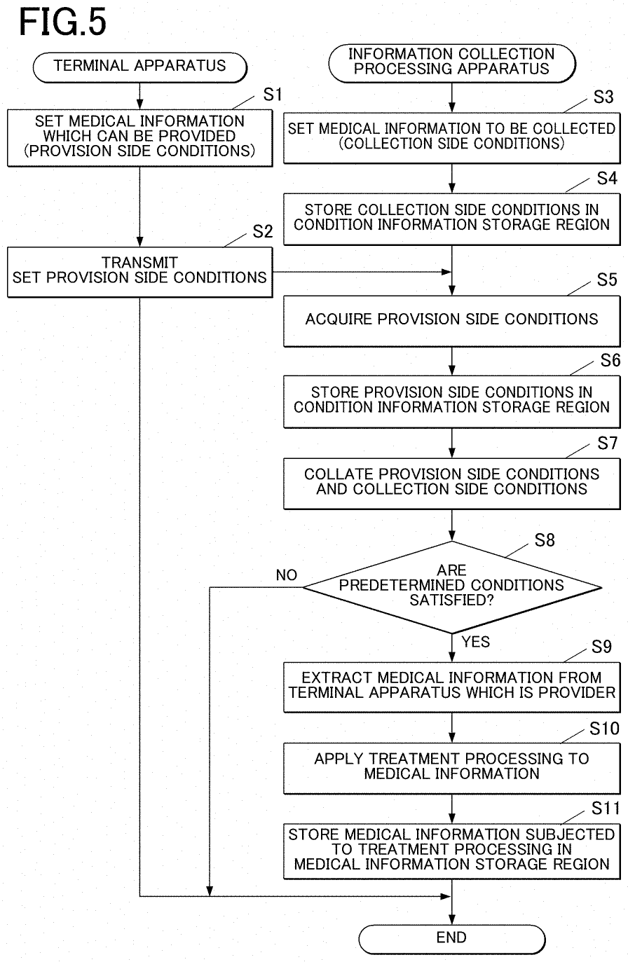

Processing to be executed by the information collection processing apparatus 1 in the present embodiment will be described next with reference to FIG. 5.

As illustrated in FIG. 5, at the terminal apparatus 2 within the facility H, the medical information which can be provided to an external apparatus, or the like, is set as the provision side conditions (step S1).

The set provision side conditions are transmitted to the information collection processing apparatus 1 (step S2). Note that the provision side conditions do not have to be directly transmitted to the information collection processing apparatus 1, and, for example, the provision side conditions may be uploaded to cloud, or the like, in a state where the provision side conditions can be accessed by the information collection processing apparatus 1.

Meanwhile, on a side of the information collection processing apparatus 1, the medical information to be collected is set as the collection side conditions (step S3). The set collection side conditions are stored in the condition information storage region 61 of the information storage 6 (step S4). Note that, as described above, the collection side conditions are not limited to those set at the information collection processing apparatus 1, and may be set in advance by default upon shipment. In this case, the collection side conditions are stored in the condition information storage region 61 of the information storage 6 since the apparatus is shipped.

If the information collection processing apparatus 1 is connected to the terminal apparatus 2 (or cloud, or the like, to which the provision side conditions are uploaded from the terminal apparatus 2), the control device 5 of the information collection processing apparatus 1 acquires the provision side conditions (step S5) and causes the provision side conditions to be stored in the condition information storage region 61 of the information storage 6 (step S6).

The information extractor 52 then collates the provision side conditions and the collection side conditions (step S7) and judges whether or not predetermined conditions are satisfied (step S8). In the case where the predetermined conditions are not satisfied (step S8: No), because the information extractor 52 cannot extract medical information, the processing is finished.

Meanwhile, in the case where the predetermined conditions are satisfied (step S8: Yes), the information extractor 52 extracts medical information which satisfies the conditions from the terminal apparatus 2 which is a provider of the medical information (step S9).

As described above, the predetermined conditions may be exact match between the provision side conditions and the collection side conditions or may be partial match.

As an extraction method by the information extractor 52, the method specified as the collection side conditions is used.

If the medical information is extracted, the information processor 53 applies treatment processing on the medical information (step S10). Note that, as the treatment processing to be performed by the information processor 53, processing specified as the collection side conditions is used.

If the treatment processing is completed, the information processor 53 causes the medical information subjected to the treatment processing to be stored in the medical information storage region 62 of the information storage 6 (step S11).

Note that the information collection processing to be performed by the information collection processing apparatus 1 is not limited to that in the order illustrated in FIG. 5.

For example, in the case where the medical information is extracted by the information extractor 52, the medical information may be caused to be stored in the medical information storage region 62 of the information storage 6 without any treatment processing being performed. The treatment processing by the information processor 53 only has to be performed until the medical information is taken outside of the facility H, and unprocessed medical information may be stored in the medical information storage region 62 until then.

As described above, there is a case where image photographing is additionally performed or a new diagnosis result is output for the same patient also after the medical information is extracted by the information extractor 52, and it is preferable to associate these kinds of additional information with the extracted medical information.

Therefore, it is preferable to maintain information necessary for associating respective pieces of medical information, such as, particularly, a patient ID, while the medical information is located within the facility H, and the information processor 53 only has to perform treatment processing of deleting a patient ID, or the like, at a timing at which the medical information is taken outside of the facility H.

In this case, all the treatment processing by the information processor 53 may be performed at a timing at which the medical information is taken outside of the facility H, or treatment processing such as reduction of image data, in which information necessary for associating the respective pieces of medical information such as a patient ID is not deleted, may be performed before the medical information is caused to be stored in the medical information storage region 62, and only treatment processing of deleting a patient ID, or the like, may be performed at a timing at which the medical information is taken outside of the facility H.

By this means, it is possible to efficiently collect medical information until the medical information is taken outside of the facility H, and it is possible to make the medical information in a safe state in which personal information from which it is possible to specify an individual patient is deleted, when the medical information is taken outside of the facility H.

As described above, according to the present embodiment, the information collection processing apparatus 1 includes an information extractor 52 which extracts medical information from the terminal apparatus 2 which is a provider which possesses medical information including a plurality of pieces of supplementary information, on the basis of the collection side conditions in which medical information to be collected at the information collection processing apparatus 1 is set and the provision side conditions in which medical information which can be provided is set, an information processor 53 which performs treatment processing including processing of deleting part of information of at least one piece of the supplementary information among a plurality of pieces of the supplementary information included in the medical information extracted by the information extractor 52, and an information storage 6 which stores the medical information.

By this means, it is possible to collect medical information which suits collection purpose and application on an information requesting side, at the information storage 6 of the information collection processing apparatus 1 in a safe state where the medical information can be taken outside of the facility H.

Further, in the present embodiment, the medical information to be stored in the information storage 6 is information regarding patients including image data and diagnosis information which can be acquired at medical institutions.

Therefore, it is possible to appropriately collect image data to be used for machine learning, or the like, to support diagnostic imaging, at the information collection processing apparatus 1.

Further, in the present embodiment, the information processor 53 can delete personal information from the medical information extracted by the information extractor 52 to process the medical information to generate data which does not include personal information.

Therefore, also in the case where a large amount of medical information is collected assuming that the medical information is taken outside of the facility H, individual patients cannot be identified and specified from the medical information, so that it is possible to realize a high-security information collection processing apparatus 1.

Further, in the present embodiment, in the case where personal information such as a patient ID is deleted from the medical information, the information processor 53 can associate the respective pieces of medical information regarding the same patient with each other by providing unique identification information (identifying indicators), or the like, which does not link to an individual patient, the facility H, or the like, to the medical information.

Therefore, it is possible to associate the respective pieces of medical information regarding the same patient with each other while enabling high-security collection of medical information which can prevent identification and specification of an individual patient, so that it is possible to collect a large amount of useful medical information.

Further, in the present embodiment, the information processor 53 may convert medical information of a provider into an irreversible characteristic amount through learning within the facility H on the information provision side.

By this means, the medical information collected by the information collection processing apparatus 1 becomes high-security medical information from which it is not possible to identify and specify an individual patient, and it is possible to collect and store a wider and a larger amount of medical information with a small data amount than in the case where image data, or the like, is stored without being processed.

Further, the information processor 53 of the present embodiment can reduce image data in the case where medical information includes the image data.

A data amount of the image data is likely to increase, and, if the image data is stored without being processed, it is necessary to increase data capacity of the information storage 6. Concerning this point, by reducing the image data, it is possible to suppress a data amount even in the case where a wide and large amount of medical information is collected and stored, so that it is possible to reduce load on the information storage 6.

Further, in the present embodiment, the information processor 53 can determine whether or not to reduce image data or a reduction rate in accordance with image characteristics of the image data.

Because there are various kinds of image data as the medical information, there is also a case where, if the image data is uniformly reduced, such reduction does not suit collection purpose of the medical information at the information collection processing apparatus 1. Concerning this point, in the case where whether or not to reduce the image data or a reduction rate is determined in accordance with the image characteristics as in the present embodiment, it is possible to collect appropriate medical information which suits collection purpose while suppressing a data amount as small as possible.

Further, in the case described above, the image characteristics may be determined on the basis of the lesion information.

In this case, it is possible to easily determine whether or not to reduce image data or a reduction rate only by referring to electronic health record information, or the like, so that it is possible to collect appropriate medical information which suits collection purpose while suppressing a data amount as small as possible.

Further, in the present embodiment, the information processor 53 may be able to cut out only data regarding a lesion position specified in accordance with the lesion information, and, when only data regarding the lesion position is cut out by the information processor 53, the information storage 6 may be able to store data regarding the lesion position.

In this case, it is possible to accumulate only necessary information in the medical information storage region 62 of the information storage 6, so that it is possible to suppress a data amount.

Further, by cutting out only necessary information, it is possible to accumulate data from which it is further difficult to specify an individual patient in the medical information storage region 62 of the information storage 6.

Still further, in the case where the information processor 53 performs treatment processing with reference to the lesion information acquired from a diagnosis result by a doctor or a result obtained in the computer-aided diagnosis system, it is also possible to specify necessary information and cut out the necessary information more appropriately.

By this means, it is possible to collect appropriate medical information which suits collection purpose while suppressing a data amount as small as possible.

Further, in the present embodiment, the information extractor 52 selects and extracts only desired medical information.

Therefore, it is possible to efficiently collect appropriate medical information which suits collection purpose while suppressing a data amount to be accumulated in the medical information storage region 62 of the information storage 6 as small as possible.

Particularly, in the case where the medical information is image data, the desired medical information is favorable data in which there is no defect in photographing conditions and positioning upon acquisition of the image data.

Therefore, because medical information in which there is a defect in photographing conditions or positioning is not extracted by the information extractor 52, it is possible to efficiently collect appropriate medical information which suits collection purpose while suppressing a data amount to be accumulated in the medical information storage region 62 of the information storage 6 as small as possible.

Note that, while the embodiment of the present invention has been described above, the present invention is not limited to such an embodiment, and it goes without saying that various modifications are possible within a range not deviating from the gist of the present invention.

For example, while, in the above-described embodiment, a case has been described where the medical information is image data (medical image data), supplementary information of the image data, patient information, or the like, the medical information is not limited to that including image data.

For example, the medical information may be data of various kinds of sound obtained through auscultation, or the like, (such as, for example, cardiac sound and cardiac murmur obtained through auscultation of the breast, carotid bruit, breath sound, or the like, abdominal vascular murmur obtained through abdominal auscultation, gurgling sound and speech (voice)), or may be various kinds of waveform data (such as, for example, time-series information of a brain waveform and a waveform of an electrocardiogram, or the like). Further, the medical information may be formed with only text data such as electronic health record information and diagnosis information input by a doctor, or the like.

Also in these cases, the information extractor 52 extracts medical information including a plurality of pieces of supplementary information from the terminal apparatus 2 which is a provider on the basis of the collection side conditions in which medical information to be collected at the information collection processing apparatus 1 is set, and the provision side conditions in which medical information which can be provided at the terminal apparatus 2 which is a provider is set, and the information processor 53 performs treatment processing including processing of deleting part of information such as personal information which is at least one piece of supplementary information among the plurality of pieces of supplementary information included in this medical information. In this case, a method of the treatment processing by the information processor 53 is selected as appropriate in accordance with types of the medical information.

Although embodiments of the present invention have been described and illustrated in detail, the disclosed embodiments are made for purposes of illustration and example only and not limitation. The scope of the present invention should be interpreted by terms of the appended claims.

What is claimed is:

1. An information collection processing apparatus comprising:
a hardware processor which extracts medical information from a terminal apparatus which possesses the medical information including a plurality of pieces of supplementary information on a basis of a collection side condition in which medical information to be collected is set and a provision side condition in which medical information which can be provided is set, and performs treatment processing of deleting at least one piece of the supplementary information among a plurality of pieces of the supplementary information included in the extracted medical information; and
an information storage which stores the medical information subjected to the treatment processing.

2. The information collection processing apparatus according to claim 1,
wherein medical information to be stored in the information storage includes image data.

3. The information collection processing apparatus according to claim 1,
wherein the hardware processor deletes the supplementary information including personal information.

4. The information collection processing apparatus according to claim 1,
wherein the hardware processor associates respective pieces of the medical information regarding a same patient with each other.

5. The information collection processing apparatus according to claim 1,
wherein the hardware processor converts medical information of a provider into an irreversible characteristic amount through learning within a facility on an information provision side.

6. The information collection processing apparatus according to claim 2,
wherein the hardware processor reduces the image data.

7. The information collection processing apparatus according to claim 6,
wherein the hardware processor determines whether or not to reduce the image data and a reduction rate in accordance with an image characteristic of the image data.

8. The information collection processing apparatus according to claim 7,
wherein the image characteristic is determined on a basis of lesion information.

9. The information collection processing apparatus according to claim 1,
wherein the hardware processor cuts out only data regarding a lesion position specified in accordance with lesion information, and
when only the data regarding the lesion position is cut out by the hardware processor, the information storage stores the data regarding the lesion position.

10. The information collection processing apparatus according to claim 1,
wherein the hardware processor performs treatment processing with reference to lesion information acquired from a diagnosis result by a doctor or a result obtained in a computer-aided diagnosis system.

11. The information collection processing apparatus according to claim 1,
wherein the hardware processor selects and extracts only desired medical information.

12. The information collection processing apparatus according to claim 1,
wherein, in a case where the medical information is image data, the desired medical information is favorable data in which there is no defect in a photographing condition and positioning upon acquisition of image data.

13. An information collection processing method comprising:
extracting medical information from a terminal apparatus which possesses the medical information including a plurality of pieces of supplementary information on a basis of a collection side condition in which medical information to be collected is set and a provision side condition in which medical information which can be provided is set;
performing treatment processing including processing of deleting at least one piece of the supplementary information among a plurality of pieces of the supplementary information included in the medical information extracted in the information extraction; and
storing the medical information subjected to the treatment processing.

14. A non-transitory computer readable recording medium storing a program causing a computer of an information collection processing apparatus which collects medical information to implement:
an information extraction function of extracting the medical information from a terminal apparatus which possesses the medical information including a plurality of pieces of supplementary information on a basis of a collection side condition in which medical information to be collected is set and a provision side condition in which medical information which can be provided is set;
an information processing function of performing treatment processing including processing of deleting at least one piece of the supplementary information among a plurality of pieces of the supplementary information included in the medical information extracted by the information extraction function; and
an information storage function of storing the medical information subjected to the treatment processing.
